(19) United States
(12) Patent Application Publication
(10) Pub. No.: US 2018/0199527 A1
(43) Pub. Date: Jul. 19, 2018

(54) PLANT

(71) Applicant: Arnoldus Cornelis Josef AMMERLAAN, Enkhuizen (NL)

(72) Inventor: Arnoldus Cornelis Josef AMMERLAAN, Enkhuizen (NL)

(21) Appl. No.: 15/742,804

(22) PCT Filed: Jul. 8, 2016
(86) PCT No.: PCT/NL2016/050506

§ 371 Date: Jan. 18, 2018

(30) Foreign Application Priority Data
Jul. 8, 2015 (NL) 2015116

(51) Int. Cl. A01H 1/02; A01H 5/08

(52) U.S. Cl. CPC A01H 1/02; A01H 5/08

(57) ABSTRACT

Described is a plant, derived from Solanum lycopersicum, the ripe fruits of which comprise a total mineral content of sodium, potassium, calcium, phosphor, manganese, magnesium, iron, copper and zinc of at least 4500 mg per kg ripe fruit. The ripe fruit also has a total solubilised sugar content of at least 5.5 w/w %, based on the total weight of the ripe fruit and optionally a lycopene content of more than 5 mg per 100 g ripe fruit.

The invention relates to a novel plant, derived from Solanum lycopersicum, the ripe fruits of which having elevated mineral and sugar level and may surprisingly have very intensive and attractive aberrant tastes.

Solanum lycopersicum, or the tomato plant, is commonly known and has many varieties. These varieties differ from one another in many aspects, such as form, colour and taste of the fruits, the tomatoes. In this respect, a tomato variety Heinz3402 has been described to have a high mineral content (Tedeschi e.a., J. Food Comp. Anal. 24 (2011) 131-139). This mineral content is however obtained by feeding the plants with a very mineral rich aqueous growth medium.

In the search for plants with new fruit trades, the inventor has developed new tomato plants that are characterised in that the ripe fruits thereof comprise a total mineral content of sodium, potassium, calcium, phosphor, manganese, magnesium, iron, copper and zinc of at least 4300 mg per kg ripe fruit, and having a total content solubilised sugars of at least 5.5 w/w %, based on the total weight of the ripe fruit. These have been obtained by crossing tomato plants with various species of the genus Solanum, followed by development of a plurality of subsequent generations by self pollination and/or crossing and/or backcrossing with a parental plant of a Solanum species or sub species, wherein in one or more generations a selection has been made on frost tolerance of the fruits thereof at a temperature of −1° C.

The content of sodium, potassium, calcium, phosphor, manganese, magnesium, iron, copper and zinc, herein also referred to as “mineral content” or “salt content” is determined in accordance with the present NEN norm EN15763, determined by inductively coupled plasma mass spectrometry (ICPMS), starting from tomato pulp, i.e. the whole fruit.

“Plants”, derived from “Solanum lycopersicum” are meant to be plants having at least one parental line of Solanum lycopersicum, that has been crossed with Solanum lycopersicum or with another Solanum species, in particular Solanum physalis, Solanum pimpinellifolium, or sub species thereof. Subsequently, stable lines are obtained from the said crossing by crossing, self pollination or backcrossing, as is known by the skilled person. Preferably, after obtaining offspring of the above mentioned first crossing, the F1, crossings are performed with Solanum lycopersicum or sub species or varieties thereof, or backcrossings are performed with one of the parental lines of a previous crossing. During the generation of new generations, at least for one generation such as e.g. F2, F3 and/or F4, or a later generation, a selection is made on frost tolerance of the fruits thereof. “Frost tolerance” is intended to mean herein that the ripe fruit of the tomato plant after being exposed at a temperature of −1° C. or lower or during at least one hour, preferably for 2, 3, 4, 5, 6, 7, 8 hours or longer does not freeze, i.e. meaning that the ripe fruit remains soft and maintains the same consistency of the fruit would have above the freezing point, e.g. at 4° C. or 10° C. Preferably a plurality of generations, such as 2 or 3 generations are subjected to selection for frost tolerance. To this end, e.g. complete intact plants carrying ripe fruits, or picked fruits can be subjected to frost.

Surprisingly, it was succeeded to obtain tomato plants of which the ripe fruits have a hitherto unknown high combined content of minerals and soluble sugars and as a result of which have a very attractive taste, which taste is maintained when the ripe fruit is stored at cool conditions, e.g. at 4° C. Ripe tomato fruits from the state of the art are almost tasteless upon cooling, which taste is only expressed after the tomatoes regained ambient temperature. The ripe fruits of the plants according to the invention can as well be regarded as tomatoes, although the plant may have a parental line of another Solanum species. The taste of the fruits can very strongly by variation of the relative mass ratio of the abovementioned elements (sodium, potassium, calcium, phosphor, manganese, magnesium, iron, copper and zinc), and is also dependent on the Solanum species, in particular of Solanum lycopersicum or sub species of varieties thereof, with which the plant according to the invention can be obtained by “back” crossing. This way, tomatoes of a special and aberrant very attractive taste have been obtained, enabling tastes to be obtained that can be associated with e.g. that of cherries, mandarins, gooseberries or watermelon. The fruits of plants according to the invention can have, per kg ripe fruit, a total content of the abovementioned elements of at least 4500 mg, 4600 mg, 4700 mg, 4800 mg, 4900 mg, 5000 mg, 5100 mg, 5200 mg, 5300 mg, 5400 mg, 5500 mg or more, up to as much as 6000 mg, 6500 mg, 7000 mg, 7500 mg or higher.

In a special embodiment, a plant is provided as described above, the ripe fruit of which comprising a total magnesium content of at least 140 mg, 150 mg, 160 mg, 170 mg, 180 mg, 190 mg, 200 mg, 210 mg, 220 mg, 230 mg, 240 mg, preferably at least 250 mg, 260 mg or 270 mg per kg ripe fruit. Magnesium is a taste determining element. By a high magnesium content in the fruits of the plants according to the invention, interesting and unexpected taste sensations are obtained as described above.

In another special embodiment, a plant as described above, is provided, the ripe fruit of which comprises a total potassium content of at least 3700 mg, 3800 mg, preferably at least 3900 mg per kg ripe fruit. Potassium is an important taste determining element as well. Accordingly, it could have been established that ripe (tomato) fruits with such a potassium content have a taste at 4° C. as if some pepper and salt would already have been added. Sodium contents of 4200 mg, 4300 mg, 4400 mg, 4500 mg per kg ripe fruit are possible.

Accordingly, a further special embodiment provides a plant as described above, the ripe fruit of which comprises a total iron content of at least 8 mg, preferably of at least 9 mg per kg ripe fruit. The iron content can be as high as 16 or 17 mg or more, up to 18 or more or 20 mg or more.

Apart from an elevated mineral content, also an elevated content solubilised sugars has been found in the fruits of the plants according to the invention. In a further special embodiment a plant is provided as described above, the ripe fruit of which has a total solubilised sugar content of at least 6 w/w %, based on the total weight of the ripe fruit. The total sugar content of a plant of the invention can in particular be more than 7 w/w %, and even up to 8 to 9 w/w %, based on the total weight of the ripe fruit. The total sugar content is preferably formed by the glucose and fructose content.

To obtain fruits of the plants of the invention having said high mineral content and high sugar content, it is important that the nutrients, including minerals are present in the nutrition or the nutrition substrate on which the plant grows, and that the plants are kept at a regular temperature of e.g. 18-21° C. The nutrition substrate can be any nutrition substrate as known to the skilled person, such as e.g. free soil, potting soil or e.g. a substrate, based on rock wool or glass wool, wherein a growth or culture medium is comprised. It has been observed that fruits with high mineral content and high sugar content according to the invention can be obtained without increasing the nutrient feed, in particular the mineral feed, above normal values. An EC value (a value for the conductivity of water, a measure for the mineral content therein, using e.g. the H198312 EC meter, Hanna Instruments, Netherlands, according to the instructions of the manufacturer) of the water that is given to the plants of 2.5 mS/cm is sufficient to obtain said high mineral content and said high sugar content in the fruits. This is remarkable, as such fruits are also formed when the plants are kept on a growth medium having an EC value of 6 mS/cm or lower. The EC values mentioned herein are expressed in mS/cm and are measured at 22° C. unless indicated otherwise.

In still another special embodiment, a plant is provided as described above, the ripe fruit of which having a Brix value of at least 12, preferably at least 14, more preferably at least 15 and most preferably at least 16. The Brix value is a measure for the content solubilised matter in the ripe fruit and indicates the mass percentage solids of the fruit. The value is expressed in the grease and can be determined by measuring the refractive index of the liquid, obtained from the fruit pulp, using a refractometer. Pulp is understood to comprise the fruits being processed to pulp, the composition of which substantially corresponds with that of intact fruits before being processed to puree/pulp. The Brix value is primarily determined by the content of solubilised sugars, in particular fructose and glucose.

Hitherto, no tomatoes having a Brix value of more than 14 are known. Such tomatoes, having a Brix value of 12 or more can only be obtained by extremely increasing the nutrient content, in particular the mineral content, in the nutrition, e.g. up to an EC value of 4. As a result of the high sugar content of the fruits of the plants of the invention, tomatoes can be obtained having hitherto unknown high Brix values, this being possible by watering with an aqueous nutrient medium having an EC value of less than 4, less than 3.5, 3.0, preferably of about 2.5 (i.e. 2.4-2.6; see Example 2 for an example of such a medium). Even an aqueous medium having an EC value of less than 2.5 is possible for watering the plants according to the invention, still resulting in the provision of fruits having the abovementioned mineral and sugar content. In a special embodiment, a plant of the invention is provided, the ripe fruits of which having a Brix value of 15 or higher. Brix values of up to 16, 17 or higher are possible.

It has also been observed that plants according to the invention as described above can be provided having an elevated lycopene content. To this end, the invention provides a plant as described above, the ripe fruits of which comprise more than 5 mg, more than 6 mg, preferably more than 7 mg, more preferably more than 8 mg, even more preferably more than 9 mg and most preferably more than 10 or 11 mg lycopene per 100 gram ripe fruit. Lycopene contents of 12 mg, 13 mg or 14 mg or more are possible. Lycopene is a carotenoid conferring red colour to the fruit. In addition, lycopene has a known medicinal activity, e.g. against cancer, in particular prostate cancer, and has bone growth promoting activity, or can be used as agent for maintaining healthy bones and as agent against cardiovascular diseases.

As already described, plants of the invention are obtained by subjecting at least fruit carrying plants of one or more generations from which the plant is generated to a selection of −1° C. or lower, while observing whether the fruit freezes or not. Plants having unfrozen fruits are selected for further generations. This does not necessarily mean that the fruits of a plant according to the invention need to be frost tolerant. By subsequent crossings and backcrossings without further selection on frost tolerance, a plant according to the invention can carry fruits that are not further frost tolerant. However, the selection on frost tolerance at an earlier generation lead to the plants being capable to carry fruits having the abovementioned high element content. However, a preferred embodiment of the invention provides a plant, the ripe fruit of which does not freeze upon exposure at a temperature of −1° C. Such a plant maintained frost tolerance upon generation of the next generation whether or not applying one or more new rounds of selection. Preferably, the plant, of which the ripe fruits do not freeze when exposed at a temperature of −1° C., are kept on a nutrition substrate having an EC value of 6 mS/cm or lower and is preferably fed with an aqueous nutrient medium having an EC value of 2.5 mS/cm, measured at 22° C.

It was surprisingly observed that plants of the invention have an increased salt tolerance as well and can grow, maintain itself and being capable to carry ripe fruits on a nutrition substrate having an EC value of 8 or higher, even of 10 or higher or of about 12. “About 12” is intended to mean an EC value of 11.5 to 12.5, in particular of 11.6 to 12.4, more in particular of 11.7 to 12.3, even more in particular of 11.8 to 12.2. Accordingly, a special embodiment provides a plant, being capable to grow on a nutrition substrate and being capable to form ripe fruits, preferably ripe fruits as defined above, the said nutrition substrate having an EC value of 8 or higher, in particular 10 or higher or having even an EC value of about 12.

In particular, plants are provided according to the invention, being derived from a crossing of a plant of the species Solanum lycopersicum with a plant of the genus Solanum, chosen from the species physalis, pimpinellifolium, lycopersicum or sub species thereof, or offspring of said crossing. Particularly, by crossing Solanum lycopersicum with the same species or with said other species S. physalis, S. pimpinellifolium, plants are obtained by further crossing, self pollination and backcrossing, that carry fruits with the abovementioned high element contents.

As crossing, self pollination and backcrossing is usually preferably performed with sub species or varieties of Solanum lycopersicum, the genetic identity of the plants of the invention is identical to that of Solanum lycopersicum in a high degree. Therefore, plants according to a special embodiment of the invention have a genetic identity of at least 85% with Solanum lycopersicum. Herein, “a plant, derived from Solanum lycopersicum” particular intends to mean a plant having at least 85% genetic identity with Solanum lycopersicum, preferably at least 90%, more preferably at least 95%, even more preferably at least 98% or 99%. When a plant according to the invention is obtained by merely crossing, backcrossing and self pollination of Solanum lycopersicum it goes without saying that the plant will then have a genetic identity of 100% to Solanum lycopersicum.

As discussed above, a plant according to the invention is in particular obtainable by the steps of:

a. crossing of a plant of the species Solanum lycopersicum with a plant of the genus Solanum, chosen from the species physalism, pimpinellifolium, lycopersicum or sub species thereof,

b. generating a first generation offspring thereof (F1) and

c. generating a plurality of subsequent generations by self crossing and/or crossing and/or backcrossing with a parental plant of a Solanum species or sub species,

d. selecting of one or more generations on frost tolerance of the fruits thereof at a temperature of −1° C., as parental plant for the generation of a next generation.

The selection on frost tolerance is e.g. performed in F3 and/or F4, but can also be performed earlier or later.

In particular, the plant is obtainable by the steps of:
i. generating a second generation offspring (F2) by self pollination of F1,
ii. generating a third generation offspring (F3) by self pollination of F2,
iii. selecting plants with frost tolerant fruit from F3 and generating of a
iv. fourth generation offspring (F4) by self pollination of the plants, selected in this step,
v. generating a fifth generation offspring (F5) by self pollination of F4,
vi. generating a sixth generation offspring (F6) by self pollination of F5,
vii. generating of a seventh generation offspring (F7) by crossing of F5 with F6.

As a result of the crossings, varieties of Solanum lycopersicum can e.g. be used of which the fruits or plants have a particular desired trade, such as taste intensity, productivity (weight of the fruit formed per plant during the season), fruit colour and fruit form which trade can be crossed into a rental ancestor of the plant of the invention or in a plant of the invention, such as resistancy against a particular disease, form and colour of the fruit (tomato), etc. The plant according to the invention can as well be a modified plant, e.g. in case one of the parental or crossed in lines are genetically modified, or when one or more of the generations on which the plant of the invention is based, has become contaminated with genetically modified material.

The above step iv. preferably comprises selecting plants having frost tolerant fruits from F4 and generating a fifth generation offspring (F5) by self pollination of the plants, selected in this step.

In another aspect, the invention provides offspring of the plant according to the invention. With “offspring” of a plant, all plants are meant to be encompassed, that are derived from said plant over one or more generations, e.g. by further crossing with other plants, by cutting or self pollination and any other propagation method, known to the skilled person. Therefore, offspring does not necessarily have to be genetically identical to the plant from which it is derived.

The invention further relates to a fruit of a plant according to the invention, in particular a tomato. As already described, the invention provides fruits, tomatoes, with a high mineral content and new and surprising characteristics, in particular with regard to taste and frost tolerance.

The invention further relates to parts of a plant according to the invention, such as leaves, stems, flowers, as well as seeds thereof.

Below, the invention is exemplified here below by examples.

### EXAMPLES

### Example 1. Generation of Frost Tolerant Tomatoes

Pollen of the first flowers of the first bunches of the species Solanum lycopersicum, S. pimpinellifolium, S. lycopersicum were jointly connected and this pollen mixture was applied to the pitstils of the first flowers of the first bunch of a Solanum lycopersicum by the use of a brush. The seeds obtained were seeded in the ground. The obtained plants (F1) were subjected to a selection on health at the end of the season, i.e. after the last fruits were formed and ripened. The above took place in a non-heated glass greenhouse in the Netherlands, in particular in October-November. The most healthy looking plants were cultured further for several generations (F2, F3) by self pollination and these were subjected to the same selection, as well as for fruits with a taste, being aberrant from the usual tomato taste. For example, a selection was performed for more intensive tastes. Fruit carrying plants of F3 were at the end of the season subjected to a temperature of −1° C., and plants, the fruits of which were not frozen were selected and further crossed by self pollination (F4, F5 and further). Starting from the F4, backcrossings were performed with Solanum lycopersicum, and selections were made on health at the end of the season, taste of the fruit, productivity and resistancy against illnesses and plagues. In F6 and F7, stable lines were found that carried fruits with high sugar and mineral contents.

By crossing F6 and F7 with other SA. lycopersicum lines the fruits of which had a pronounced taste, plants were obtained with vary surprising and aberrant tastes, and it is believed that this is a result of the high mineral content and possibly also the high sugar content of the fruits of said parental lines, derived from F6 and F7. This way, a plurality of plants were obtained with fruits having a salty taste or a taste mixture of salt and pepper, as well as a plurality of plants with extremely sweet tasting fruits, the fruits showing variation in size, form and weight. Accordingly, a plurality of plants were found that carried fruits of 50-200 gram with an aberrant taste, such as a sweet taste.

### Example 2. Analysis of Ripe Fruits

Of two thereof, lines 14-706 and 13-710, analysis of the pulp/puree of ripe fruits was performed by SGS, CTS-Agri Laboratory, Spijkenisse, Netherlands, determining the sugar content by HPLC analysis, and the elements by ICPMS according to NEN norm NEN-EN15763. See the table. These analyses are shown in the table. Values of reference tomatoes are derived from the website http://nevo-onlin.rivm.nl of the Dutch National Institute for Public Health and the Environment. To this end, lines 14-706 and 13-710 were kept on a nutrition substrate of potting soil of Van Egmond Potgrond B.V., Amsterdam, Netherlands (50 v/v % German garden peat RHP, 50 v/v % Baltic bog moss peat 0-20) enriched with 40 kg Florisol Profi (Stephan Schmidt KG, Domburg, Duitsland), 6 kg chalk (Kalkwerke Breckweg, Rheine, Germany) per 1000 litres, having an EC value of 6.0 mS/cm, and fed with a standard aqueous nutrient medium having an EC value of 2.5 (9 mM nitric acid, 3 mM phosphoric acid, 12 mM potassium lye, 8.5 mM calcium nitrate, 4 mM magnesium sulphate, 1.5 mM calcium chloride, 40 μM iron, 20 μM manganese, 20 μM zinc, 65 μM boron, 1.5 μM copper and 1 μM molybdene). Both EC values were measured at 22° C. using a HI98312 EC meter, Hanna Instruments, Netherlands.

### Example 3. Salt Tolerance

Potting soil from Example 2 was mixed with 1 kg NaCl per 20 litres, resulting in an EC value of the soil of 12. A plurality of juvenile plants according to the invention, including B13-710 were planted in the potting soil and allowed to grow in an unheated glass house wherein the temperature ranged between about 12-25° C. and a air humidity of about 50%. The plants were fed daily with the standard nutrient medium having an EC value of 2.5 from Example 2. The plants grew to mature fruit carrying plants, and the mineral and sugar contents, as well as the lycopene content in the obtained ripe fruits corresponded significantly with the values as measured in Example 2.

The above plants were also obtained by treating the pistils of S. lycopersicum with pollen of the separate species separately.

TABLE

| | Invention | | Reference tomatoes (NWE/NEVO) | | | | | |
|---|---|---|---|---|---|---|---|---|
| | A | B | Normal | Cherry | Mean | Bunch | Meat | |
| | Supersweet | Salty | tomato | tomato | tomato | tomato | tomato | Mean |
| | 14-706 | 13-710 | raw | raw | raw | raw | raw | NEVO |
| Sugars % | Brix 16 | Brix 12 | Brix 6 | Brix 8 | Brix 6 | Brix 6 | Brix 4 | Brix 6 |
| Glucose | 3.84 | 2.97 | 1.3 | 1.8 | 1.4 | 1.3 | 1.1 | 1.38 |
| Fructose | 4.08 | 3.04 | 1.6 | 2.1 | 1.6 | 1.5 | 1.3 | 1.62 |
| Lactose | 0 | 0 | 0 | 0 | 0 | 0 | 0 | 0 |
| Sacharose | 0 | 0 | 0 | 0.1 | 0 | 0 | 0 | 0.02 |
| Maltose | 0 | 0 | 0 | 0 | 0 | 0 | 0 | 0 |
| Total Sugars | 7.9 | 6.01 | 2.9 | 4 | 3.1 | 3.1 | 2.4 | 3.1 |
| Elements mg/kg | | | | | | | | |
| Sodium | 20 | 21 | 20 | 30 | 20 | 10 | 20 | 20 |
| Potassium | 3900 | 4100 | 2460 | 3430 | 2660 | 2620 | 2120 | 2658 |
| Calcium | 96 | 68 | 110 | 70 | 90 | 80 | 90 | 88 |
| Phosphor | 100 | 730 | 230 | 330 | 260 | 260 | 230 | 262 |
| Manganese | 3.5 | 2.5 | | | | | | |
| Magnesium | 370 | 270 | 80 | 110 | 80 | 80 | 70 | 84 |
| Iron | 16 | 9.9 | 3 | 3 | 4 | 2 | 7 | 3.8 |
| Cupper | 1.1 | 0.82 | 0.4 | 0.3 | 0.3 | 0.2 | 0.3 | 0.3 |
| Zinc | 6.8 | 4.2 | 1.5 | 1.2 | 1.6 | 1 | 2.7 | 1.6 |
| Total Elements | 4513.4 | 5206.42 | 2904.9 | 3974.5 | 3115.9 | 3053.2 | 2540 | 3117.7 |
| β-Carotene mg/100 gr | | 0.317 | 0.464 | 0.771 | 0.531 | 0.408 | 0.482 | 0.5312 |

1. Plant derived from Solanum lycopersicum,
wherein the plant produces ripe fruits
comprising a total mineral content of sodium, potassium, calcium, phosphor, manganese, magnesium, iron, copper and zinc of at least 4500 mg per kg ripe fruit, and
having a total solubilised sugar content of at least 5.5 w/w %, based on the total weight of the ripe fruit, or fruit thereof.

2. Plant according to claim 1 or fruit thereof, the ripe fruit of which comprises, per kg ripe fruit:
a total content of magnesium of at least 140 mg, preferably 200 mg, more preferably at least 250 mg, and/or
a total potassium content of at least 3800 mg, and/or
a total iron content of at least 8 mg, preferably at least 9 mg.

3. Plant according to claim 1 or fruit thereof, the ripe fruit of which having a total solubilised sugar content of at least 6 w/w %, based on the total weight of the ripe fruit.

4. Plant according to claim 1 or fruit thereof, the ripe fruit of which having a Brix value of at least 12, preferably at least 14, more preferably at least 15, most preferably at least 16.

5. Plant according to claim 4 or fruit thereof, the ripe fruit of which having a Brix value of 18 or less.

6. Plant according to claim 1 or fruit thereof, the ripe fruit of which comprising more than 5 mg lycopene per 100 g ripe fruit.

7. Plant according to claim 1 or fruit thereof, the ripe fruit of which comprises, per 100 g ripe fruit, more than 6 mg, preferably more than 7 mg, more preferably more than 8 mg, even more preferably more than 9 mg, and most preferably more than 10 mg lycopene.

8. Plant according to claim 1 or fruit thereof, the ripe fruit of which comprises said mineral and sugar content when the plant is kept on a nutrient medium having an EC value of 6 or lower and is fed with an aqueous nutrient medium having an EC value of 2.5.

9. Plant according to claim 1 or fruit thereof, the ripe fruit of which does not freeze when exposed to a temperature of −1° C.

10. Plant according to claim 1 or fruit thereof, being capable to grow on a nutrition substrate and to form ripe fruits, preferably ripe fruits as defined in any of the preceding claims, which nutrition substrate has an EC value of 8 or higher, 10 or higher or an EC value of about 12.

11. Plant according to claim 1, obtainable by the steps of:
a. crossing of a plant of the species Solanum lycopersicum with a plant of the genus Solanum, chosen from the species physalis, pimpinellifolium, lycopersicum or sub species thereof,
b. generating a first generation offspring thereof (F1) and
c. generating a plurality of generations by self crossing and/or backcrossing with a parental plant of a Solanum species or sub species,
d. selecting one or more generations on frost tolerance of the fruits thereof at a temperature of −1° C., as parental plant for the generation of a next generation, or offspring thereof, or fruit thereof.

12. Plant according to claim 11, comprising the steps of:
i. generating a second generation offspring (F2) by self pollination of F1,
ii. generating a third generation offspring (F3) by self pollination of F2,
iii. selecting plants with frost tolerant fruits from F3 and generating a fourth generation offspring fourth generation offspring (F4) by self pollination of the plants, selected in this step,
iv. generating a fifth generation offspring (F5) by self pollination of F4,
v. generating a sixth generation offspring (F6) by self pollination of F5,
vi. generating of a seventh generation offspring (F7) by crossing of F5 with F6, or offspring thereof, or fruit thereof.

13. Plant according to claim 12 or offspring thereof, or fruit thereof, wherein step iv. comprises selecting plants with frost tolerant fruits from F4 and generating a fifth generation offspring (F5) by self pollination of the plants, selected in this step.

14. Plant according to claim 1 or fruit thereof, having 85% genetic identity with Solanum lycopersicum, preferably at least 90%, more preferably at least 95%, even more preferably at least 98% or 99%.

15. (canceled)

16. Method for the preparation of a plant according to claim 1, comprising the steps of:
a. crossing of a plant of the species Solanum lycopersicum with a plant of the genus Solanum, chosen from the species physalis, pimpinellifolium, lycopersicum or sub species thereof,
b. generating a first generation offspring thereof (F1) and
c. generating a plurality of generations by self crossing and/or backcrossing with a parental plant of a Solanum species or sub species,
d. selecting one or more generations on frost tolerance of the fruits thereof at a temperature of −1° C., as parental plant for the generation of a next generation.

17. Method according to claim 15, comprising the steps of:
i. generating a second generation offspring (F2) by self pollination of F1,
ii. generating a third generation offspring (F3) by self pollination of F2,
iii. selecting plants with frost tolerant fruits from F3 and generating a fourth generation offspring fourth generation offspring (F4) by self pollination of the plants, selected in this step,
iv. generating a fifth generation offspring (F5) by self pollination of F4,
v. generating a sixth generation offspring (F6) by self pollination of F5,
vi. generating of a seventh generation offspring (F7) by crossing of F5 with F6.

18. Method according to claim 16, wherein step iv. comprises selecting plants with frost tolerant fruits from F4 and generating a fifth generation offspring (F5) by self pollination of the plants, selected in this step.
